(19) United States
(12) Patent Application Publication
(10) Pub. No.: US 2025/0011852 A1
(43) Pub. Date: Jan. 9, 2025

(54) METHODS AND DEVICES FOR USING AIRFLOW-DRIVEN, EVAPORATIVE GRADIENTS

(71) Applicant: THE REGENTS OF THE UNIVERSITY OF CALIFORNIA, Oakland, CA (US)

(72) Inventors: Yu-Hwa Lo, San Diego, CA (US); Edward Wang, San Diego, CA (US); Zhilin Guo, La Jolla, CA (US); Rui Tang, San Diego, CA (US)

(21) Appl. No.: 18/695,332

(22) PCT Filed: Sep. 28, 2022
(86) PCT No.: PCT/US2022/077204

§ 371 Date: Mar. 25, 2024

Related U.S. Application Data
(60) Provisional application No. 63/249,518, filed on Sep. 28, 2021.

(51) Int. Cl. C12Q 1/6851; B01L 3/00; C12Q 1/70; G01N 33/543

(52) U.S. Cl. CPC C12Q 1/6851; B01L 3/5023; C12Q 1/701; G01N 33/54386; B01L 2300/069; B01L 2300/10

(57) ABSTRACT

Microfluidic paper-based analytical methods and devices are disclosed. In one implementation, a method includes applying a gas flow to a first spot of a porous or fibrous membrane for fluorescent detection assay to generate an enriched substance underneath the first spot in the porous or fibrous membrane, controlling the gas flow such that the enriched substance causes a fluorescent intensity change in the porous or fibrous membrane, and performing a fluorescent detection readout based on the fluorescent intensity change.

### CROSS-REFERENCE TO RELATED APPLICATION

This patent document claims priority to and benefits of U.S. Provisional Appl. No. 63/249,518, entitled “METHODS AND DEVICES FOR USING AIRFLOW-DRIVEN, EVAPORATIVE GRADIENTS” and filed on Sep. 28, 2021. The entire contents of the before-mentioned patent application are incorporated by reference as part of the disclosure of this document.

### STATEMENT REGARDING FEDERALLY SPONSORED RESEARCH OR DEVELOPMENT

This invention was made with government support under 2020-20090400002 awarded by The Intelligence Advanced Research Projects Activity (IARPA). The government has certain rights in the invention.

### TECHNICAL FIELD

The invention relates to microfluidic paper-based analytical devices.

### BACKGROUND

Microfluidic paper-based analytical devices have long been established as an attractive area for point-of-care testing. These devices use paper as the key platform in the fabrication of various diagnostic assays Paper, comprised primarily of cellulose fibers, presents an inexpensive and lightweight material which is beneficial for overall cost reduction. However, there are limitations on the sensitivity and capability of the microfluidic paper-based analytical devices in handling complex assays.

### SUMMARY

The disclosed technology can be implemented in some embodiments to provide an airflow-based, evaporative method that is capable of manipulating fluid flows within paper membranes to offer new functionalities for multistep delivery of reagents and improve the sensitivity of microfluidic paper-based analytical devices (uPADs).

In some implementations of the disclosed technology, a method includes applying a gas flow to a first spot of a porous or fibrous membrane for fluorescent detection assay to generate an enriched substance underneath the first spot in the porous or fibrous membrane, controlling the gas flow such that the enriched substance causes a fluorescent intensity change in the porous or fibrous membrane, and performing a fluorescent detection readout based on the fluorescent intensity change.

In some implementations of the disclosed technology, a device includes a pad including a central portion that includes a liquid and a constituent therein, wherein an evaporation rate of the liquid increases with a gas flow over the central portion, wherein the constituent has a lower evaporation rate than the liquid under the same gas flow rate, and a nozzle positioned to allow one or more gases to pass through and positioned at a distance above the central portion of the pad.

In some implementations of the disclosed technology, a device includes a hollowed pad including a hole portion at a center of the pad and a replenishing pad portion surrounding the hole portion, and a nozzle structured to allow one or more gases to pass through and positioned at a distance above the hollowed pad.

In some implementations of the disclosed technology, a device includes a fibrous membrane including a portion that includes a liquid and a constituent therein, wherein an evaporation rate of the liquid increases with a gas flow over the portion, wherein the constituent has a lower evaporation rate than the liquid under the same gas flow rate, a nozzle positioned to allow one or more gases to pass through and positioned at a distance above the central portion of the pad, and a sensor configured to detect a color change of an area under a flow of the one or more gases.

In some implementations of the disclosed technology, a device includes a fibrous membrane, a liquid in the fibrous membrane, wherein the evaporation rate of the liquid increases with a gas flow, a constituent in the liquid that is moved towards a position of the gas flow, and a nozzle positioned to introduce a gas flow to a segment of the fibrous membrane.

In some implementations of the disclosed technology, a device includes a fibrous membrane including a central area, a replenishing area connected to the central area to allow liquid flow from the replenishing area to the central area, and a nozzle positioned above the central area to direct an air flow to the central area, wherein the liquid flow includes a constituent that is moved towards a position of the air flow.

The above and other aspects and implementations of the disclosed technology are described in more detail in the drawings, the description and the claims.

### BRIEF DESCRIPTION OF THE DRAWINGS

FIG. 1 shows schematic for the inward, radial replenishment of fluid and evaporation by ambient and airflow induced methods based on some implementations of the disclosed technology.

FIG. 2 shows schematic of the airflow-based enrichment for the concentration of colorimetric substrates produced during on-paper ELISA.

FIG. 3 shows schematic of the airflow-based enrichment for the concentration of colorimetric substrates produced during on-paper ELISA.

FIG. 4A shows a profile plot of pixel intensity for enriched and non-enriched samples of dye deposited on paper. FIG. 4B shows an image sequence of dye localization in paper with hydrophobically patterned barriers indicated by black outlines.

FIG. 5 shows an image sequence for the sequential delivery of colored dyes by moving the airflow spot on a paper device patterned with PDMS channels.

FIG. 6A shows a 3D-schematic of a point-of-care (POC) device used for sequential delivery. FIG. 6B shows an example of sequential delivery using dye as indicators. FIG. 6C shows schematic mapping the initial reagent spots for the multistep immunoassay.

FIG. 7A shows plots of intensity ratio v. copy number between red and grayscale, green and grayscale, and green and red pixels. FIG. 7B shows before and after enrichment photos corresponding to different initial RNA copy numbers of the COVID-19 genome.

FIG. 8A shows a comparison of non-enriched and enriched intensity plots for IgG concentrations from 67 fM to 6.7 nM. FIG. 8B shows respective images from enriched and non-enriched samples from a single experimental set.

FIG. 9 shows an example of a setup during airflow enrichment.

FIG. 10 shows RT-qPCR results for isolated SARS-COV-2 RNA.

FIG. 11 shows an example of RT-LAMP colorimetric readout system for on-paper RT-LAMP products before and after enrichments.

FIG. 12 shows an image signal analysis process for RT-LAMP colorimetric readout.

FIG. 13 shows an example of 3D-printed ELISA plate.

FIG. 14 shows an example method of colorimetric detection assay based on some embodiments of the disclosed technology.

### DETAILED DESCRIPTION

Disclosed are methods, materials, articles of manufacture and devices that pertain to an airflow-based, evaporative method that is capable of manipulating fluid flows within paper membranes to offer new functionalities for multistep delivery of reagents and improve the sensitivity of microfluidic paper-based analytical devices (uPADs) by 100-1000 times.

The disclosed technology can be implemented in some embodiments to apply an air-jet to a pre-wetted membrane, generating an evaporative gradient such that any solutes become enriched underneath the air-jet spot By controlling the lateral position of this spot, the solutes in the paper strip are enriched and follow the air jet trajectory, driving the reactions and enhancing visualization for colorimetric readout in multistep assays. The disclosed technology can be implemented to drive the sequential delivery in multistep immunoassays as well as improve sensitivity for colorimetric detection assays for nucleic acids and proteins via loop-mediated isothermal amplification (LAMP) and enzyme linked immunosorbent assay (ELISA). For colorimetric LAMP detection of the COVID-19 genome, enrichment of the solution on paper could enhance the contrast of the dye in order to more clearly distinguish between the positive and negative results to achieve a sensitivity of 3 copies of SARS-Cov-2RNAs. For ELISA, enrichment of the oxidized tetramethylbenzidine (TMB) substrate yielded a sensitivity increase of two-to-three orders of magnitude when compared to non-enriched samples-having a limit of detection of around 200 fM for IgG. Therefore, this enrichment method represents a simple process that can be easily integrated into existing detection assays for controlling fluid flows and improving detection of biomarkers on paper.

Microfluidic paper-based analytical devices (uPADs) are foundational devices for point-of-care testing, yet suffer from limitations in regards to their sensitivity and capability in handling complex assays. The disclosed technology can be implemented in some embodiments to provide an airflow-based, evaporative method that is capable of manipulating fluid flows within paper membranes to offer new functionalities for multistep delivery of reagents and improve the sensitivity of uPADs by 100-1000 times. This method applies an air-jet to a pre-wetted membrane, generating an evaporative gradient such that any solutes become enriched underneath the air-jet spot. By controlling the lateral position of this spot, the solutes in the paper strip are enriched and follow the air jet trajectory, driving the reactions and enhancing visualization for colorimetric readout in multistep assays. The technique has been successfully applied to drive the sequential delivery in multistep immunoassays as well as improve sensitivity for colorimetric detection assays for nucleic acids and proteins via LAMP and ELISA. For colorimetric LAMP detection of the COVID-19 genome, enrichment of the solution on paper could enhance the contrast of the dye in order to more clearly distinguish between the positive and negative results to achieve a sensitivity of 3 copies of SARS-Cov-2RNAs. For ELISA, enrichment of the oxidized TMB substrate yielded a sensitivity increase of two-to-three orders of magnitude when compared to non-enriched samples-having a limit of detection of around 200 fM for IgG. Therefore, this enrichment method represents a simple process that can be easily integrated into existing detection assays for controlling fluid flows and improving detection of biomarkers on paper.

Microfluidic paper-based analytical devices (uPADs) have long been established as an attractive area for point-of-care testing (POCT). These devices use paper as the key platform in the fabrication of various diagnostic assays. Paper, comprised primarily of cellulose fibers, presents an inexpensive and lightweight material which is beneficial for overall cost reduction. In addition, the presence of cellulose fibers allows the capability of wicking liquids via capillary action, thereby allowing simple operation without the use of external pumps. These advantages coupled with the biocompatibility and flexibility in diagnosing a variety of biomarkers have accelerated the growth of these devices as diagnostic tools.

Within this field, one of the most prominent readout methods for uPADs has been through colorimetric means. In colorimetric assays, detection of a biomarker is accompanied by a color change which is often proportional to the amount of biomarker present. Color change can be induced by various detection chemistries such as small-molecule organic indicators, metal nanoparticles, and chromogenic enzyme reactions. While the results can be either qualitative or quantitative, colorimetric assays represent a significant portion of current uPAD research due to their user-friendly nature.

Despite this abundance of research in colorimetric detection, these assay's suffer from key limitations which have slowed down its commercialization. The frontmost of these issues remains in their poor sensitivity, having detection limits orders of magnitudes lower than those performed by clinical instruments. Sensitivity issues are often attributed to the inhomogeneous distribution of the colorimetric substance and the presence of background noise generated by either the paper or sample itself. Various methods to resolve these issues within colorimetric assays have been proposed. Here, novel means of color generation and amplification have been the most widely studied alternatives. These include the use of enzyme-nanoparticle conjugates, hybridized metallic and organic nanoparticles, and enhanced metallic nanoparticle staining which act to further increase color development and subsequent contrast.

In addition to poor sensitivity, lack of fluid control in rudimentary uPADs has restricted their use to single reaction type assays. As paper based ELISAs are typically more complex and require the stepwise delivery of various reagents, multiple mechanisms of fluid flow control have been explored. Within this field, two categories of techniques have emerged-passive and active means of fluid control. Generally, passive methods manipulate fluid flow through physical restructuring or chemical treatment of the device. These include the use of dissolvable solutes, creation of physical valves, and geometric adjustments. While passive methods are advantageous regarding ease of fabrication, design simplicity, and expandability, they have limitations in fluid manipulation as the speed and direction of fluid flow is determined by pre-set elements in the device. Conversely, active methods aim to control fluid flow with the incorporation of external inputs. Using mechanical actuation and geometric transformations, various types of switches and valves have been designed to manage fluid flow speed, direction, or entry. Based on their programmable nature, active methods can therefore offer a higher degree of flexibility and more precise control over fluids.

While the limitations regarding immunoassay sensitivity and fluid control have been subject to numerous studies, these two issues remain somewhat exclusive as the rationale to improve these constraints is either to increase the limit of detection or for integration into multistep assays, respectively. The disclosed technology can be implemented in some embodiments to provide an active, airflow-based enrichment method that can control the fluid flow and improve sensitivity for paper-based colorimetric assays. The method based on some embodiments of the disclosed technology includes aiming an airflow nozzle perpendicular to a pre-wetted membrane. A differential evaporative gradient is induced as the area immediately underneath the airflow experiences a higher evaporation rate than the rest of the membrane. Due to the formation of this gradient, the surrounding liquid will migrate towards the spot in order to replenish the lost water During this process, the replenishing water will carry any dissolved molecules towards this area such that they will be concentrated underneath the nozzle. While solute enrichment occurs while the airflow source is stationary, fluid control and sequential delivery can be achieved by dynamic airflow positioning across the filter surface.

### Modification of Airflow Temperature to Increase Speed of Enrichment

Modification of the physical parameters of the airflow can influence the rate of evaporation and the kinetics of enrichment. Therefore, adjustments to the airflow speed and temperature allow additional degrees of freedom when conducting and optimizing assays. Increases in both these parameters can also serve to reduce process times needed for the completion of the assay.

The disclosed technology can be implemented in some embodiments to provide the simplicity and flexibility of the airflow enrichment method through its applications in colorimetric, paper-based protein and nucleic acid detection. The disclosed technology can be implemented in some embodiments to use paper-based ELISA and isothermal RT-LAMP as these assays show the most potential for being integrated into widespread POCT devices. For protein detection, evaporative-induced fluid control is incorporated into a multistep immunoassay as a delay valve and switch to demonstrate the sequential delivery of specific reagents. In addition, the airflow enrichment of the colorimetric substrate generated in ELISA and isothermal RT-LAMP could be focused on a single spot to improve sensitivity and visualization without the need for alternative systems of color generation.

### Detection of Chemical (Small Molecule) Analytes

In addition, the extension of sensitivity and visualization improvements can be applied in the detection of inorganic molecules. Colorimetric assays which identify the presence of heavy metals, sugars, pesticides, etc. can also be subject to concentration due to the ubiquitous nature of the enrichment meebanism. Performing enrichment and detection for these inorganic molecules share the same principle as demonstrated before, by establishing evaporative gradients to drive the colorimetric substance to a defined area.

Addition of fluorescence, chemifluoresence, etc. enrichment accommodating different solvents.

The mechanism behind airflow enrichment may be further expanded to accommodate a variety of different assay types and solvents Regarding assay type, additional assays which do not utilize colorimetric reporters such as fluorescence, chemiluminescence, and electrochemical based assays can be subject to enrichment in improving overall sensitivity. Furthermore, while the enrichment studies were performed on biological macromolecules in aqueous solutions, non-aqueous, volatile solvents are also compatible with the enrichment method. The flexibility of the enrichment method thereby makes it applicable to a multitude of already existing paper-based assays.

### Evaporative Enrichment

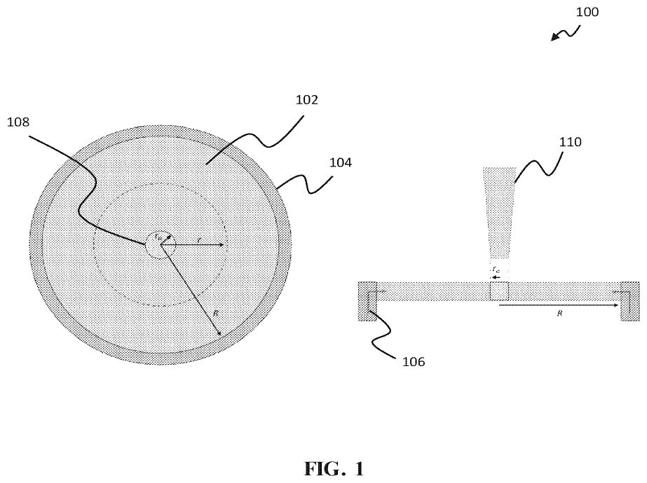

FIG. 1 shows schematic for the inward, radial replenishment of fluid and evaporation by ambient and airflow induced methods based on some implementations of the disclosed technology.

Previous mathematical models have been developed which examine how physical and geometric variables factor into wicking behavior when evaporation is considered. The disclosed technology can be implemented in some embodiments to provide a system that includes a circular slice of porous media (102) to make peripheral contact with an unlimited reservoir of fluid. At the perimeter of the porous slice (104), the capillary force induces the inward, radial penetration of liquid towards its center (108). At the center, a nozzle is positioned above the porous slice and generates a flow of air (106) that is constrained within the nozzle radius. To model the system as a two-dimensional problem using planar coordinates, it is assumed that the thickness of the slice is much smaller than the radial dimensions.

During the enrichment process, two methods of evaporation occur on the wetted surface of the porous slice. The first is the ambient evaporation that is not affected by airflow, and the second is the airflow-induced evaporation. We therefore consider the total evaporation rate, K, which is the volume of evaporated liquid per area and time (cm3/m2·s) and is the sum of the ambient evaporation (Kₒ) and the airflow-induced evaporation (K₁):

$$K ( r ) = {K}_{o} + {K}_{1} u ( {r}_{o} - r ) \qquad Eq . ( 1 )$$

where rₒ is the radius of the airflow jet and u represents a unit step function. Here, it is assumed that all evaporation will only occur on the top surface of the filter and that the airflow speed above the filter surface is negligible outside rₒ.

When the steady state is reached and the enriched area under the nozzle has not completely dried, the continuity equation can be written as

$$2 π rK ( r ) = - \frac{dF ( r )}{dr} \qquad Eq . ( 2 )$$

where F refers to the volumetric flux of water per time (cm³/s)

In order to drive the flux, Darcy's law for liquid flow can be expressed as:

$$\frac{F}{A} = - \frac{S}{η} \frac{dP}{dr} \qquad Eq . ( 3 )$$

where the flux, F, of liquid flow is through a cylindrical surface having area A=2πrhγ, S is the permeability of the porous medium, γ is the porosity of the membrane, η is the liquid viscosity, and P is the pressure of the liquid. Substituting the cylindrical area into equation 3 and solving for the volumetric flux yields,

$$F = - \frac{2 π h γ S}{η} r \frac{dP}{dr} \qquad Eq . ( 4 )$$

Substituting Eq. (4) into Eq. (2) and combining with Eq. (1), we obtain

$$[ {K}_{o} + {K}_{1} u ( {r}_{o} - r ) ] = \frac{h γ S}{η} [ \frac{{d}^{2} P}{{dr}^{2}} + \frac{1}{r} \frac{dP}{dr} ] \qquad Eq . ( 5 )$$

The boundary conditions can be defined as:

$$P ( R ) = {P}_{c} ,$$

$$\frac{dP}{dr} {\vert}_{r = 0} = 0 ( see Supplmentary for this BC )$$

where P_{c} is the capillary pressure having the form:

$${P}_{c} = \frac{2 σ cos {θ}_{S}}{{R}_{eff}} \qquad Eq . ( 6 )$$

where σ is the surface tension, θs is the contact angle formed between solid and liquid, and R_{eff} is the effective pore radius of the porous medium.

$$P ( r ) = \frac{- η}{4 h γ S} ( {K}_{o} ) ( {R}^{2} - {r}_{o}^{2} ) - \frac{η}{4 h γ S} ( {K}_{o} + {K}_{1} ) ( {r}_{o}^{2} - {r}^{2} ) + {P}_{c} \qquad Eq . ( 7 )$$

$$0 \leq r \leq {r}_{o}$$

$$P ( r ) = \frac{- η}{4 h γ S} ( {K}_{o} ) ( {R}^{2} - {r}^{2} ) + {P}_{c}$$

$$R \geq r \geq {r}_{o}$$

P(r)≥0 for all r. When P(r)=0 at a certain r, the region is dried up.

$$F ( {r}_{o} ) = - \frac{2 π h γ S}{η} r \frac{dP}{dr} {\vert}_{{r}_{0}} = π {r}_{0}^{2} ( {K}_{o} + {K}_{1} ) \qquad Eq . ( 8 )$$

In some implementations, when the wetted porous slice undergoes evaporation, it experiences varying degrees of water saturation. Therefore, the relation can be:

$$P ( r ) = {P}_{c} n ( r ) \qquad Eq . ( 9 )$$

$$0 \leq n ( r ) \leq 1$$

where n is the fraction of the pores (capillaries) that are completely saturated with water.

Integrating to find the water volume within rₒ, the region under the air flow,

$$V ( {r}_{o} ) = 2 π h γ \text{?} n ( r ) rdr = π h γ {r}_{o}^{2} - \frac{π η {r}_{o}^{2}}{8 {P}_{c} S} [ {K}_{o} ( 2 {R}^{2} - {r}_{o}^{2} ) + {K}_{1} {r}_{o}^{2} ] \qquad Eq . ( 10 )$$

$$\text{?} \text{indicates text missing or illegible when filed}$$

The ratio between the volume from the water flux and the volume under the air flow determines the enrichment factor, a value which characterizes how effectively solutes are transported and concentrated under the airflow spot:

$$Eq . ( 11 )$$

$$EF ( enrichment factor ) = \frac{F ( {r}_{o} ) t}{V ( {r}_{o} )} = \frac{( {K}_{o} + {K}_{1} ) t}{h γ + \frac{η}{8 {P}_{c} S} [ {K}_{1} {r}_{o}^{2} - {K}_{o} ( 2 {R}^{2} - {r}_{o}^{2} ) ]}$$

As the enrichment method utilizes a rapid jet of low humidity air to promote evaporation, we have operated under the condition K₁r₀²>>KₒR² in all cases regardless of environmental humidity and temperature. This condition can be readily achieved because wetted, porous materials develop a saturated boundary layer which can be more easily displaced with airflow. Under this condition, (11) becomes

$$EF ( enrichment factor ) = \frac{F ( {r}_{o} ) t}{V ( {r}_{o} )} \text{?} \frac{{K}_{1}}{h γ + \frac{η}{8 {P}_{c} S} {K}_{1} {r}_{o}^{2}} t \qquad Eq . ( 12 )$$

$$\text{?} \text{indicates text missing or illegible when filed}$$

Therefore, within a given time period, the variables governed by the airflow (K₁, rₒ) can be controlled to maximize the enrichment factor.

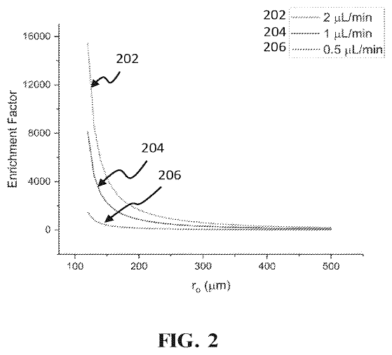

FIG. 2 shows enrichment factor v. air-jet radius for different evaporation rates after 10 minutes of enrichment.

The influence of these variables according to Eq. (11) is shown in FIG. 2, where the enrichment factor is plotted as a function of rₒ for different K₁ using tabulated values for the physical (S, η, P_{c}, γ, etc.) parameters. Because the evaporation rate for a saturated, porous material behaves like that of a free surface of water, the ambient evaporation rate is set as 0.1 uL/min for a circular slice of material with R=0.5 cm. While rₒ is a simple geometric parameter, K₁ is influenced by a variety of factors including airflow speed, air flow temperature etc. These factors are incorporated into K₁ to simplify the model, however present additional degrees of freedom when looking to further increase the enrichment factor.

### Airflow-Based Enrichment on Paper

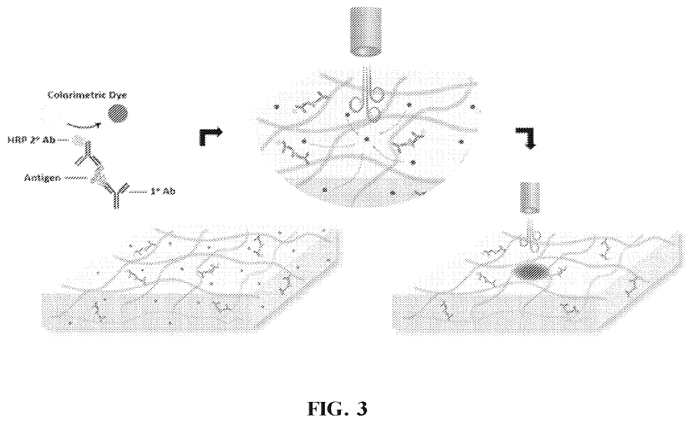

FIG. 3 shows schematic of the airflow-based enrichment for the concentration of colorimetric substrates produced during on-paper ELISA.

Specifically, FIG. 3 depicts the operation principle for airflow-based enrichment in a paper matrix, using ELISA as an example for the purpose of illustration. This technique occurs on a pre-wetted filter. A nozzle connected to a nitrogen source is brought down perpendicular to the filter, and nitrogen is blown to create an evaporative gradient by reducing the thickness of a boundary layer containing saturated water vapor immediately above the surface. While any compressed air source can accomplish this, nitrogen can be chosen as it is a “dry” (low humidity) source of airflow and gave consistent enrichment results when compared to compressed, ambient air. Due to accelerated localized evaporation by the nitrogen airflow, fluid flows driven by capillary action will converge towards the area under the jet air from the filter's periphery to replenish the lost water. In doing so, any dissolved solutes will be carried by the flow and eventually accumulate underneath the nozzle. The enrichment factor derived previously is therefore a measurement of how concentrated the solutes become and how small the accumulation spot is. The disclosed technology can be implemented in some embodiments to use this technique to enhance the visibility and sensitivity of nucleic acid and protein colorimetric detection assays—including ELISA. For these assays, the colorimetric substance can be enriched to a single spot under the nozzle to improve the uniformity and concentration in addition to easier detection.

Static or Dynamic Movement of the Airflow Nozzle Leads to Solute Enrichment and Movement

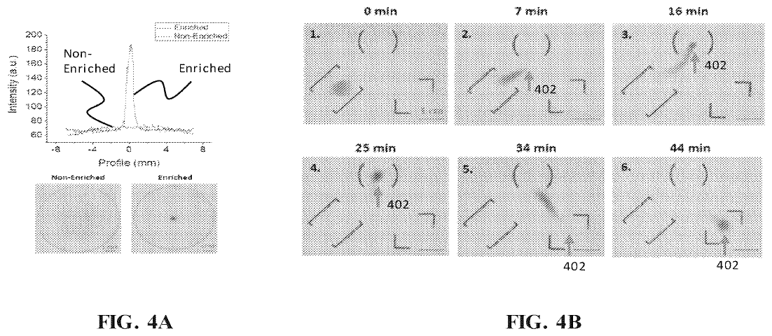

FIG. 4A shows a profile plot of pixel intensity for enriched and non-enriched samples of dye deposited on paper. Images underneath represent corresponding non-enriched (before) and enriched (after) sample. FIG. 4B shows an image sequence of dye localization in paper with hydrophobically patterned barriers indicated by black outlines. Images are taken during specified time points during dynamic movement of the nozzle. Actual nozzle position is indicated by the arrow 402.

Airflow enrichment can accommodate two modes of fluid movement depending on whether the nozzle is in a static or dynamic position. When the nozzle position remains fixed (FIG. 4A), solute enrichment occurs as evaporative-loss leads to capillary-induced replenishment from the surrounding area. To visualize this effect, 7 uL of a blue, diluted dye solution is deposited on a 16 mm diameter filter cutout. The cutout is placed concentric to a replenishing pad saturated with water, and a 200 um diameter nozzle is aimed perpendicular to the surface. Initiating the airflow establishes an evaporative gradient which generates fluid flows that are directed radially inward and transports the dye from a larger area to a defined spot. As seen in the profile plot, this three-dimensional transport can thereby enhance visualization and detection of the initially diluted dye as it becomes more uniformly concentrated and unobscured from interacting with light.

Incorporation into 3D Paperfluidics

In addition to these flows along the 2D plane, any solutes will also move and localize at the paper's surface due to evaporation being a surface-driven phenomenon. Once the water is transported near the surface and undergoes a phase change from a liquid to a gas, any dissolved molecules stay dissolved in the liquid phase and remain at the surface. Therefore, establishment of an evaporative gradient allows fluid flow manipulation in all three dimensions depending on where the nozzle is placed. This can therefore be utilized not only in conventional 2D paper-based assays, but also in 3D paper-based assays which can accommodate the performance of more complex assays.

Dynamic movement of the nozzle allows transportation of the solute to specified positions along the paper surface. The transport mechanism remains the same as the enrichment process, but the continual re-establishment of new evaporative gradients allows the direction of the replenishing flows to be controlled. In FIG. 4B, 1 uL of blue dye is deposited on a wetted filter paper with hydrophobic patterns. The patterned filter paper is placed concentric to a replenishing pad saturated with water to prevent the paper from drying out. As less emphasis is placed on the degree of enrichment and more on the speed of transport, a wider 1.5 mm diameter nozzle accompanied with higher airflow speeds are used in comparison to that of the static enrichment. As shown in FIG. 4B, the localization of the dye can be controlled simply by changing the position of the nozzle above the paper surface.

### Sequential Delivery Using the Airflow-Enrichment Method

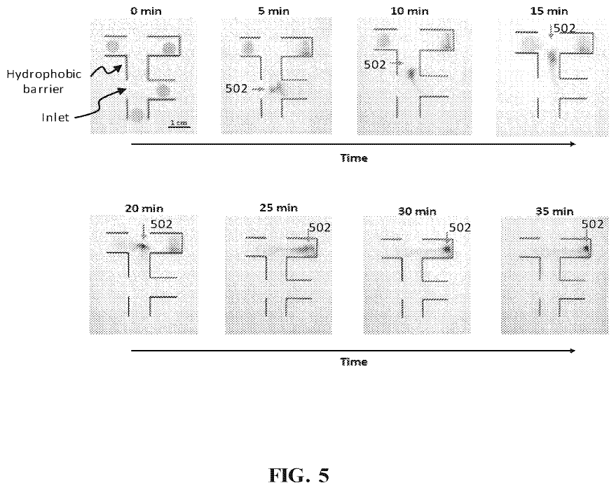

FIG. 5 shows an image sequence for the sequential delivery of colored dyes by moving the airflow spot (indicated by arrow 502) on a paper device patterned with PDMS channels. Black outlines represent the hydrophobic barriers and breaks in the outline indicate inlets to the channel design.

The disclosed technology can be implemented in some embodiments to show how controlled delivery of multiple reagents, visualized by different dye colors, can be accomplished using the airflow enrichment technique (FIG. 5). On a piece of filter paper patterned with hydrophobic channels, a combination of static and dynamic nozzle movements serves to mix and transport specified reagents, respectively. The filter paper is overlayed concentric to a replenishing pad cutout, such that contact between the two materials is only made at the paper's perimeter. Then, 1 uL of each dye color is spotted on the paper and could be mixed in a stepwise fashion based on the position of the nozzle, indicated by the arrow 502. For this setup, water is transferred from the replenishing pad to the filter paper and eventually moves towards the interior of the channel through the openings in the design. While the openings act as inlets, the movement of the nozzle governs the position of the outlet and determines where fluid flows will converge. As there is only one nozzle (outlet) in this system, additional openings are incorporated to reduce backflow and prevent early mixing of downstream reagents. For sections where the dye is to remain stationary throughout the process, such as the green dye, a closed channel is created by walling off three sides to create an area of dead volume.

The advantage of airflow-enrichment lies in the mechanism responsible for fluid control. For many designs focused on sequential delivery, the primary driving force for fluid transport is through capillary action. While capillarity is still present during airflow enrichment, it plays a secondary role with evaporation being the main driver of fluid movement. This has considerable effects when determining the overall design and costs of the assay. For one, conventional assay's that rely on capillarity require relatively large sample volumes as they must continually imbue the device with the sample or running buffer in order to reach the detection zone. For evaporative-driven transport, delivery of a small, initial volume of sample can be dragged to downstream detection zones simply by guiding it with the nozzle. In this case, the nozzle is able to transport 1 uL of the initial blue dye from beginning to end. In addition, for assays that require interactions between molecules to occur, the airflow technique allows for more efficient mixing. Rather than relying on the slower process of diffusion, the nozzle can be left in a static position such as in the 20- and 35-minute time points (FIG. 5) to facilitate the enrichment of different molecules and promote binding events. Finally, in designs that transport fluid through capillary action, the fluid flow is always “on” and specific obstructions are implemented in order to slow and control the flow rate for sequential delivery. For evaporative driven transport, the fluid flow can be turned “on” and “off” depending on whether any air is being blown on the paper surface. The lack of any obstructions keeps the fabrication process simple and reduces the amount of space needed to accommodate the obstruction itself.

### Application in a Multistep Immunoassay

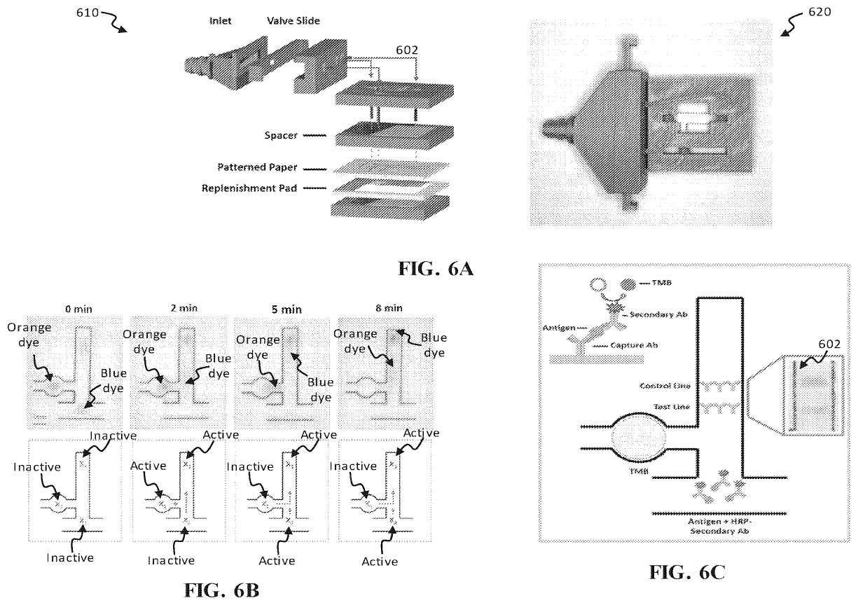

FIG. 6A shows a 3D-schematic of a point-of-care (POC) device used for sequential delivery with arrows 602 indicating tubing connections (610) and a top-down, real photo (620) of the assembled components. FIG. 6B shows an example of sequential delivery using dye as indicators. The top row shows the real photos taken at specified time points while the bottom row represents which nozzles are active and inactive as well as the strength of fluid flow from each channel (indicated by arrow). FIG. 6C shows schematic mapping the initial reagent spots for the multistep immunoassay. The box 602 is the real image of the test lines taken 32 minutes after starting the assay.

The disclosed technology can be implemented in some embodiments to incorporate the airflow enrichment method in a possible POC device. In some implementations, control of the fluid flow is dictated by the movement of a single nozzle and its relative position over the paper. As integrating a moving nozzle would be difficult in a POC device, the disclosed technology can be implemented in some embodiments to use a static arrangement of three individual nozzles (FIG. 6A) to accomplish step-wise delivery for an immunoassay. In addition, multiple nozzles are required as the dimensions of the patterned paper are reduced when compared in FIG. 3 to decrease transport distances and consequently, the time for assay completion. Because of the constrained dimensions, a single nozzle cannot facilitate step-wise delivery as the evaporation would cause replenishing flows from multiple channels in the general vicinity. Therefore, for this setup, a single source of airflow originates from a central inlet and is then split up into the three separated nozzles—each of which the airflow can be turned on or off. The combination of active and inactive nozzles determines the predominant fluid flow within the patterned paper and therefore which reagent will be delivered faster.

A demonstration of stepwise delivery as visualized with dye is shown in FIG. 6B, where the delivery of the blue dye is achieved prior to that of the orange dye through a combination of active nozzles. Within this system, the disclosed technology can be implemented in some embodiments to define two means of fluid transport-one is the evaporative mass transport caused by the airflow nozzles, and the other is the replenishment transport driven by capillarity in maintaining a constant level of water saturation in the entire membrane. For an active nozzle far from the replenishment pad and walled off such as pos. 3, the fluid has reached the terminal position and will simply evaporate off and deposit any solutes in this location. However, for activated nozzles closer to the replenishment pad such as pos. 1 and 2, the evaporation will reduce the amount of fluid entering the channel and thereby slow the net movement of the fluid. This in turn will cause the delay of fluid to enter the main channel and allow sequential delivery of reagents. Therefore, in FIG. 6B, the nozzles at X1 and X3 are first activated to delay movement of the orange dye such that the main replenishing flow is supplied by the blue channel Once the blue dye reached the end, X2 and X3 are activated to allow the main source of replenishing flow from the orange dye.

The operation principle based on some embodiments of the disclosed technology can be extended to an actual immunoassay, where 2 uL of TMB, as well as 0.5 uL of a pre-mixed solution of the antigen and the horseradish peroxidase (HRP)-conjugated secondary antibody are deposited in separate channels (FIG. 6C). Test and control lines are immobilized downstream of these reagents prior to running in PBST buffer. During the assay, the antigen-secondary antibody complex is first drawn up the channel by activating nozzles 1,3 for 10 minutes. Not only does this step first transport the antigen complex to the detection lines, but also acts as a washing step for the latter duration to remove any unbound proteins in this area. Then, the nozzles at pos. 2,3 are activated to begin moving the TMB to the detection lines Once a faint signal could be seen in the control line, the airflow is stopped, and time is given to allow further color development. The signals from these lines are clearly visible using 4 nM of the antigen after allowing the assay to run for roughly 30 minutes. Comparing the time scales for this immunoassay with the dye demonstration in FIG. 6B, the immunoassay takes much longer to complete. In some implementations, the longer duration is primarily caused by two factors: (i) the relative low solubility of the unreacted TMB which leads to a higher retention factor on paper and requires more fluid to be evaporated to move it through the channel, (ii) the high salt concentration in the PBST running buffer which, after prolonged evaporation, will crystallize in the paper and act as a barrier for further fluid migration. Additional time is therefore spent re-dissolving these barriers when the activated nozzles switch position.

### Improving Visualization on Paper for Colorimetric RT-LAMP

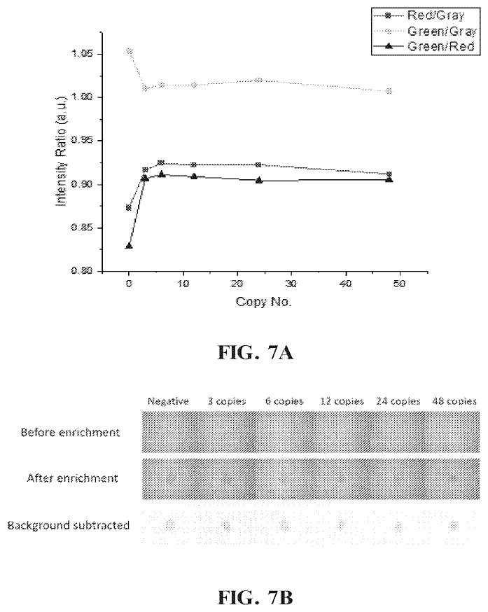

FIG. 7A shows plots of intensity ratio v. copy number between red and grayscale, green and grayscale, and green and red pixels. FIG. 7B shows before and after enrichment photos corresponding to different initial RNA copy numbers of the COVID-19 genome. FIG. 7B includes bottom “background subtracted” row to better visualize the color of each spot.

To test whether the airflow enrichment could improve visualization in already established colorimetric assays, the technique based on some embodiments can be incorporated for on paper RT-LAMP in the detection of the COVID-19 genome (FIGS. 7A-7B). Heat inactivated viral particles are spiked in nuclease-free water to the desired concentrations and then amplified using a commercially available RT-LAMP kit. For all copy numbers, amplification via LAMP is performed in test tubes for 60 minutes prior to depositing 3 μL of the LAMP product onto paper strips. The strips are connected to a replenishing flow of nuclease-free water, and the airflow is turned on for 5 minutes before imaging. As shown in FIG. 7B, comparison between the “before” and “after” enrichment demonstrates how the process drastically improves the visibility of the dye. Furthermore, the enrichment does not alter the results of the test. The pH-sensitive color of the RT-LAMP result, either red (negative) or yellow (positive), is influenced by polymerase activity which releases a proton per each incorporated nucleotide. As the enrichment involves the concentration of molecules, false positives may occur due to co-enrichment of hydronium ions. However, a large shift in the green/red pixel intensity ratio from 0 (negative control) to 3 RNA copies supports the validity of the results as the negative control remains red.

Due to the presence of buffer in the master mix solution, the final enrichment spot remains larger than previously demonstrated in FIG. 4A as early salt crystallization inhibits further condensation. While additives can be considered to reduce salt build up, we chose not to include any as it may affect the pH of the solution and therefore the accuracy of the test. Even with a larger spot size, the results of the tests are still clear to the naked eye. The ease of integrating the airflow-based enrichment to various colorimetric assays, coupled with its short process time, demonstrates the flexibility and simplicity of the technique.

### Improving On-Paper ELISA Sensitivity

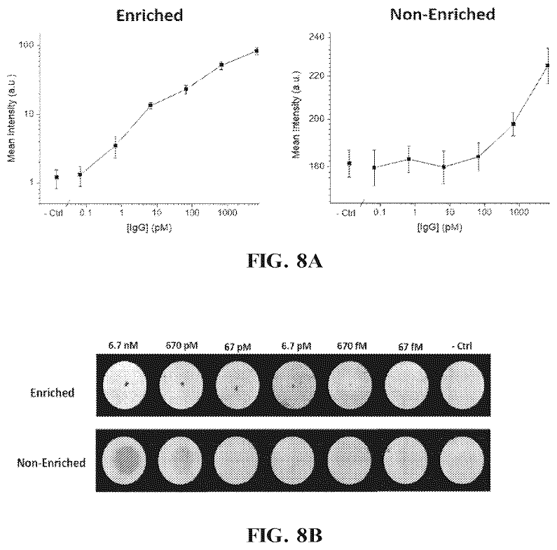

FIG. 8A shows a comparison of non-enriched and enriched intensity plots for IgG concentrations from 67 fM to 6.7 nM. Error bars are calculated from three sets of experiments. FIG. 8B shows respective images from enriched and non-enriched samples from a single experimental set. Brightness and contrast are adjusted for better visualization of the TMB product.

The disclosed technology can be implemented in some embodiments to use ELISA to model our enrichment method in improving the sensitivity for protein detection assays (FIGS. 8A-8B). While ELISA on paper has attracted multiple studies in the past, a key obstacle in detection is due to the non-uniformity and unequal distribution of the colored substrate. Therefore, achieving a high enrichment factor and being capable of transporting the substrate to a defined spot may increase the limit of detection. For this assay, TMB is chosen as the colorimetric substrate as it has been shown to elicit one of the most sensitive responses in comparison with others. However, a drawback of using TMB lies in the relative insolubility of the oxidized product in water which hinders the formation of an airflow-enriched spot. Therefore, the addition of the 0.1% glycerol (v/v) in 2M HCl “stop” solution served multiple purposes. The first is to prevent further development of the substrate during enrichment and keep the colorimetric development times consistent between the enriched and non-enriched samples. The second is to protonate the TMB product to increase its solubility for enrichment. Glycerol is also added in small amounts to prevent early salt crystallization which halts spot formation during the evaporative process. The addition of an acidic solution to the TMB product causes a color change from blue to yellow which explains the different colors between the enriched and non-enriched samples. Despite the color change, the enriched samples are able to distinguish antigen concentrations by more than 2 orders of magnitude as compared to the non-enriched samples while only spending 15 minutes under airflow. This demonstrates the case at which airflow enrichment can be integrated in current detection-based assays given the fraction of time needed to conduct the process and the much-improved detection sensitivity.

### Materials and Methods

### Colorimetric Assay Reagents

For the reverse transcription loop-mediated isothermal amplification (RT-LAMP) assay, DNA and RNA are prepared. The RT-LAMP primers against Nucleocapsid (N−1) and ORFla-1 genes are synthesized and are designed according to a similar assay. The SARS-COV-2 RNA is prepared. For ELISA, Rabbit IgG, goat anti-rabbit IgG, TMB, mouse anti-goat IgG, Ultrapure water, 25% glutaraldehyde and chitosan are prepared. Anti-rabbit IgG antibody conjugated with HRP is prepared. The blocking buffer is comprised of 0.05% v/v Tween-20 and 1% w/v bovine serum albumin (BSA) in 1× phosphate-buffered saline (PBS) The antibody incubation solution consists of the blocking buffer with the conjugated antibody at a 1:4000 ratio.

### Enrichment Setup

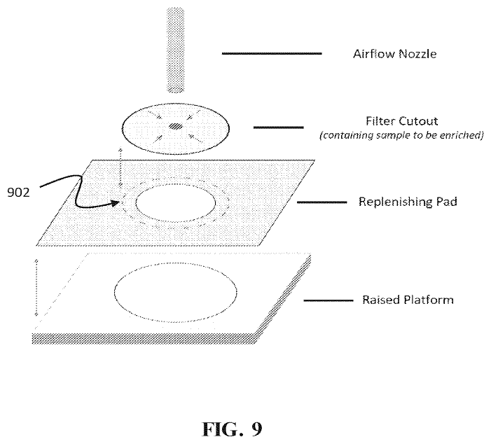

Pre-pulled, 1 mm O.D. capillaries are prepared and the tips are sanded down to roughly 200 um in diameter. A single tip is mounted on an xyz-stage through a 3D-printed holder and connected to a nitrogen source by rubber tubing. The mass flow rate is adjusted by pressure valves incorporated along the rubber tubing and specific values are determined by a mass flow meter. The specified sample is deposited on a cutout of filter paper and placed on a hollowed-out replenishing pad comprised of blotting paper saturated with water. The contact between the filter cutout and the replenishing pad only occurs at the perimeter of the filter cutout (FIG. 9). This arrangement is then placed on a 3D-printed platform that also contains a hollow section and the capillary tip is brought about 2 mm above the filter piece.

### Enrichment of Dye

The filter paper is punched into circular, 16 mm diameter cutouts and initially wetted with 10 μL of DI water. After allowing the water to spread, 3 μL of a diluted food dye solution is added to the center of the cutout which is then overlayed on top of a hollowed-replenishing pad saturated with DI water. The section is placed under the capillary tip and the nitrogen flow is turned on to a rate of 0.1 l/min. Airflow enrichment occurs for 15 minutes before imaging the section under a desktop ring-light using an iPhone 10 camera.

### Fluid Control

Patterns and outlines are designed using CAD software and printed directly on filter paper. The outlines are then traced over using a syringe containing a 10:1 ratio of PDMS to curing agent and subsequently placed in a 60° C. oven to cure for 1 hour. Prior to enrichment, the patterned filter paper is dampened with DI water and 1 uL of dye is spotted in the starting positions. The patterned filter paper is then placed on a water-saturated replenishing pad and positioned 2 mm under a 1.5 cm diameter metal nozzle with a nitrogen flow of 0.25 l/min. The relative position of the nozzle is changed by moving the platform with the filter paper.

Application into Multistep Immunoassay

The 3D models for the POC device are designed using 3D CAD software and printed. Rubber gaskets are placed in the openings and glass capillaries (1 mm O.D./0.7 mm I.D.) are inserted in the specified areas and connected with rubber tubing. The patterned paper and dye demonstration is performed in the same manner to 3.4. For the immunoassay, the channels are treated with 0.25 mg/mg chitosan (in 1% acetic acid) and allowed to dry completely before adding 2.5% glutaraldehyde and incubating for 1 hour. The treated paper is then washed in a wash bath containing 1×PBS and allowed to dry before striping the test and control lines comprised of 0.2 mg/mL of goat anti-rabbit antibody and 0.39 mg/mL of mouse anti-goat antibody, respectively. The paper is then blocked for 30 minutes and subsequently allowed to dry in a desiccator. Before performing the assay, 0.5 uL of solution containing 1:2000 reporter antibody and 4 nM rabbit IgG is deposited in one channel, and 2 uL of TMB is deposited in the other. The paper is then pre-wetted by pressing it against a saturated membrane containing PBST, before placing it on another hollowed replenishment pad also containing PBST. The pads are placed in the POCT device and the airflow is started. Images of the paper are taken with a camera.

### Enrichment of RT-LAMP Products on Paper

Colorimetric LAMP is performed by adding 1 μL of the specified copy number of isolated SARS-COV-2 RNA, 12.5 μL of colorimetric RT-LAMP 2× master mix, 2.5 μL of the 10× primer mix and diluting with nuclease free water up to 25 uL in total volume. All the solutions are prepared fresh and kept in closed test tubes to limit exposure to atmospheric CO₂. The reactions are then incubated in a 65° C. oven for 60 min. The RT-LAMP samples are then taken out and placed on ice for 30 seconds before being stored at room temperature. For the enrichment, filter paper is cut into strips and 3 uL each RT-LAMP product is deposited in the middle. Enrichment is carried out for 5 minutes with at 5 uL/min replenishing flow of nuclease-free water from syringe pumps. Before and after images are taken by a digital microscopic camera with preset image capture settings.

### RT-LAMP Colorimetric Readout Signal Analysis

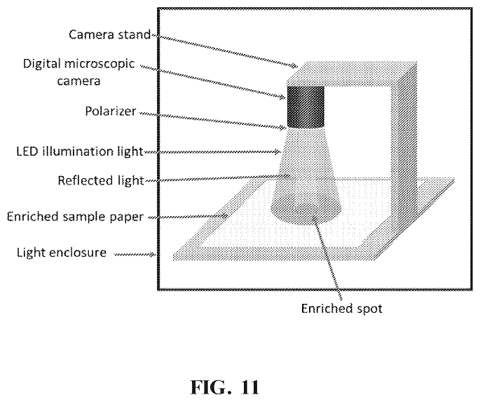

The image signal analysis process of RT-LAMP colorimetric readout is shown in FIG. 11. The effect of the GAMMA correction on the captured image is eliminated on the original image to remove the biased on the pixel intensity introduced by GAMMA correction. The original image is subtracted by the background level first to remove the noise introduced from the background. A binary mask is generated through fixed intensity thresholding and preset enrichment spot size to determine the region of interest (ROI). The processed image is then convoluted with the binary mask to exclude pixels outside of the ROI. The convoluted color image is split into single color channels, and the average pixel intensity is calculated based on the average pixel intensity of the RGB color channel images. The image processing pipeline is developed based on ImageJ and MATLAB. All enrichment sample paper is processed using the same pipeline and parameter setting.

### On-Paper ELISA

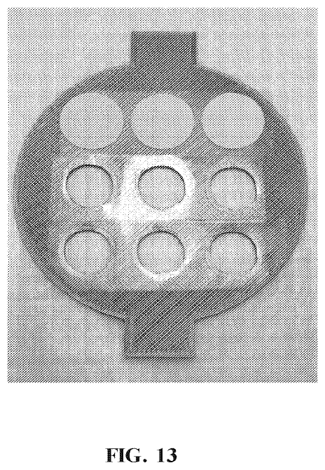

Double-sided tape is placed on a 3D-printed plate containing a 3×3 array of 12 mm diameter holes (FIG. 13). The tape is cut out around the holes and 16 mm diameter filter circles are overlayed on top with only the perimeter making contact. Enhanced antibody immobilization is performed by adding 20 uL of 0.25 mg/mg chitosan (in 1% acetic acid) to all filter circles and letting them dry completely. Then 20 uL of 2.5% glutaraldehyde in 1×PBS is added to the circles and the plate is covered with a petri dish for 1 hour to avoid evaporation Each circle is then washed four times with 20 uL of PBS with the excess liquid being removed between washes by pressing the plate against blotting paper. After washing. 10 uL of the capture antibody solution is deposited and allowed to incubate for 30 minutes. The circles are washed four times with 20 uL of PBS and successively blocked with 30 uL of blocking buffer for an additional 30 minutes. After blocking the circles are washed once with 20 uL of PBS and 3 uL of the Rabbit IgG at specified concentrations is added to each circle. This is incubated for 15 minutes before adding 7 uL of the secondary antibody solution and incubating for another 2 minutes. The circles are then removed from the plate and into a wash bath containing 0.05% Tween in 1×PBS and a separator to prevent individual circles from contacting each other. The wash bath is placed on a shaker for 30 minutes with the wash solution being changed out every 10 minutes. The circles are then stored in 1×PBS until use.

### Comparison Between Enriched and Non-Enriched Samples

For the non-enriched sample, the circles are removed from the PBS and transferred on blotting paper to remove excess liquid prior to being placed on a cleaned plate. The color development is initiated by adding 7 uL of TMB solution and allowing 80 seconds for the reaction to proceed before imaging under a desktop ring-light. A similar procedure is performed with the enriched samples except that the reaction time is shortened to 70 seconds to include a washing step afterwards. This step includes transferring the circle onto blotting paper and depositing 1 mL of ultrapure water to reduce salt levels. Then, 6 uL of 0.1% glycerol (v/v) in 2M HCl is added to the circle to stop the reaction during the enrichment process. The circle is enriched under the 200 um diameter capillary nozzle for 15 minutes until completely dry and imaged under the ring-light.

### ELISA Imaging

The images are processed by first subtracting the background by 50 pixels, then enhancing the contrast by 0.3%. The images are then split into RGB color channels and inverted. For the non-enriched samples, the selected ROI consisted of the entire filter circle diameter. In the enriched samples, a binary mask is used to highlight the enriched area and the ROI consists of a 2 mm diameter section around the highlight. After finding the mean respective mean intensities from each channel, the magnitude of the combined channels is calculated for the mean intensity.

The disclosed technology can be implemented in some embodiments to provide a simple, airflow-based method that can manipulate fluid flows in paper matrices Depending on how the method is used, enrichment or lateral transportation of solutes can be performed using minimal external equipment. The enrichment process is integrated into existing colorimetric assays for genomic and proteomic detection to significantly enhance the visualization and sensitivity of the assays. The disclosed technology can be implemented in some embodiments to accomplish multistep, sequential delivery of reagents for future assays requiring higher complexity. This method ultimately presents a unique example where sensitivity improvement and fluid control can be accomplished using a single technique. Despite focusing on colorimetric detection assays in some embodiments, the disclosed method can be easily incorporated into the broader field of mPADs.

FIG. 9 shows an example of a setup during airflow enrichment. The filter cutout for solute enrichment or movement is overlayed a water-saturated replenishing pad with roughly 5 mm of contact (902). This is placed on a hollowed, 3D-printed platform raised 25 mm from the workbench's surface.

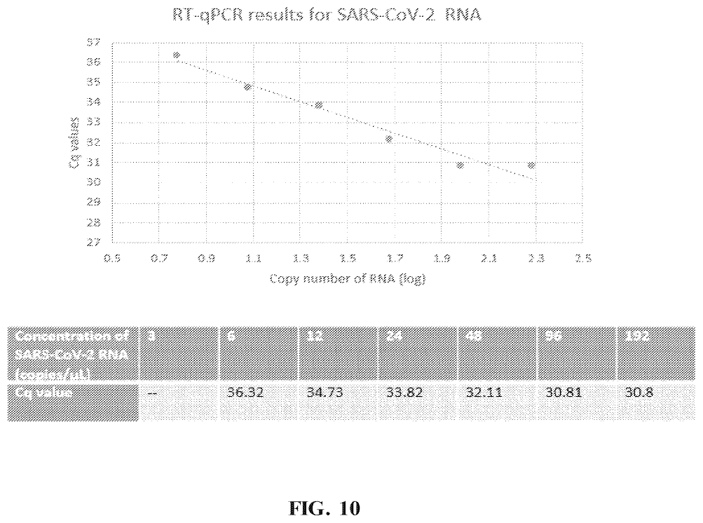

FIG. 10 shows RT-qPCR results for isolated SARS-COV-2 RNA. The Cq value for SARS-COV-2 RNA with different copy numbers: 192, 96, 48, 24, 12, 6 can be measured.

FIG. 11 shows an example of RT-LAMP colorimetric readout system for on-paper RT-LAMP products before and after enrichments.

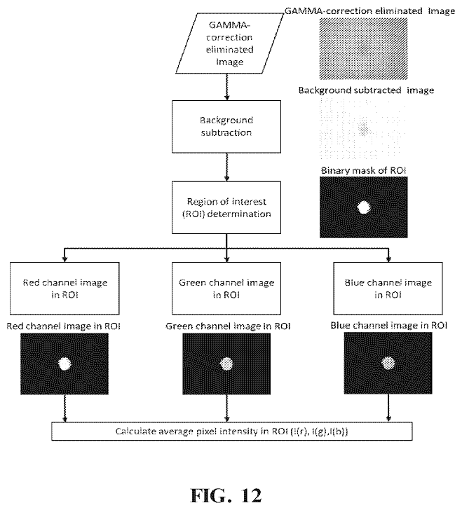

FIG. 12 shows an image signal analysis process for RT-LAMP colorimetric readout.

FIG. 13 shows an example of 3D-printed ELISA plate that can be used to secure each 16 mm-diameter filter cutout (top row) containing different concentrations of antigen. The holes in the array are 12 mm in diameter and double-sided tape can be placed over each row prior to cutting out the interior with a blade.

### Explanation of Boundary Condition in Theoretical Model

$$\frac{dP}{dr} {\vert}_{r = 0} = 0$$

For the first BC, we choose the pressure reference at r=R by assuming P(r=R)=0.

We find the 2nd BC based on the following:

For small r,

$$- π {r}^{2} ( {K}_{0} + {K}_{1} ) = ( F ( r ) - F ( 0 ) )$$

$$- π r ( {K}_{0} + {K}_{1} ) = \frac{( F ( r ) - F ( 0 ) )}{r}$$

When r→0,

$$0 = \frac{dF}{dr} {\vert}_{r = 0}$$

From Eq. (4),

$$0 = \frac{dF}{dr} {\vert}_{r = 0} = - \frac{2 π h γ S}{η} \text{?} \frac{d}{dr} ( r \frac{dP}{dr} ) = \frac{2 π h γ S}{η} \frac{dP}{dr} {\vert}_{r = 0}$$

$$\text{?} \text{indicates text missing or illegible when filed}$$

This shows the 2nd BC should be

$$\frac{dP}{dr} {\vert}_{r = 0} = 0$$

Paper-based enrichment of RT-LAMP products

TABLE 1
Primer/probe sets, and positive controls used in RT-qPCR assay

| | |
|---|---|
| Primer/probe sets | N1 primer/probe mix |
| | N2 primer/probe mix- |
| | RNase P (RP) primer/probe mix |
| Positive controls: | SARS-CoV-2 viral RNA |
| | Human specimen-RPP30 plasmid (HSC) |

TABLE 2
Concentrations of each component in master mix of RT-qPCR assay

| Component | Volume in 20 μL rxn |
|---|---|
| iTaq universal probes reaction mix (2x) | 10 μL |
| iScript RT | 0.5 μL |
| Primer/probe set | 1.5 μL |
| Nuclease-free H2O | 6 μL |

TABLE 3
The steps and thermocycler conditions for RT-qPCR experiment

| Step Temp Time | Temp | Time |
|---|---|---|
| 1-Reverse Transcription | 50° C. | 15 min |
| 2-Enzyme activation | 95° C. | 2 min |
| 3 | 95° C. | 10 sec |
| 4 | 55° C. | 30 sec |
| 5 | Read plate *FAM | |

Repeat steps 3-5 for 45 cycles.

TABLE 4
The sequence of primers against Nucleocapsid
(N-1) and ORF1a-1 genes

Nucleocapsid (N-1)
N-1-F3 -
TGGCTACTACCGAAGAGCT
N-1-B3 -
TGCAGCATTGTTAGCAGGAT
N-1-FIP -
TCTGGCCCAGTTCCTAGGTAGTCCAGACGAATTCGTGGTGG
N-1-BIP -
AGACGGCATCATATGGGTTGCACGGGTGCCAATGTGATCT
N-1-LF -
GGACTGAGATCTTTCATTTTACCGT
N-1-LB -
ACTGAGGGAGCCTTGAATACA
Orf1a-1
ORF1a-1-F3 -
CTGCACCTCATGGTCATGTT
ORF1a-1-B3 -
AGCTCGTCGCCTAAGTCAA
ORF1a-1-FIP -
GAGGGACAAGGACACCAAGTGTATGGTTGAGCTGGTAGCAGA
ORF1a-1-BIP -
CCAGTGGCTTACCGCAAGGTTTTAGATCGGCGCCGTAAC
ORF1a-1-LF -
CCGTACTGAATGCCTTCGAGT
ORF1a-1-LB -
TTCGTAAGAACGGTAATAAAGGAGC

TABLE 5
Concentrations of each oligonucleotide in the 10x primer mix

| | 10X CONCENTRATION | 1X CONCENTRATION |
|---|---|---|
| PRIMER | (STOCK) | (FINAL) |
| FIP | 16 μM | 1.6 μM |
| BIP | 16 μM | 1.6 μM |
| F3 | 2 μM | 0.2 μM |
| B3 | 2 μM | 0.2 μM |
| LOOP F | 4 μM | 0.4 μM |
| LOOP B | 4 μM | 0.4 μM |

TABLE 6
Concentrations of each reagent in RT-LAMP assay

| | | | NO-TEMPLATE | |
|---|---|---|---|---|
| | EXTRACTED RNA | | CONTROL | |
| | TARGET DETECTION | | (NTC) | |
| WarmStart Colorimetric | 12.5 | μL | 12.5 | μL |
| LAMP 2X Master Mix | | | | |
| LAMP Primer Mix (10X) | 2.5 | μL*2 | 2.5 | μL*2 |
| Isolated SARS-CoV-2 RNA | 1 | μL | — | |
| dH2O | 6.5 | μL | 7.5 | μL |
| Total Volume | 25 | μL | 25 | μL |

In some embodiments of the disclosed technology, the iTaq universal one-step RT-qPCR kit is used for the RT-qPCR process of isolated SARS-COV-2 RNA. The RT-qPCR assay targets the nucleocapsid (N) genomic regions by including 2 SARS-COV-2 specific primer/probe sets (N1, N2) and a human specimen primer/probe set (RNase P). RT-qPCR is used to monitor the amplification of DNA by measuring the fluorescence signals in real time between the thermal cycles. Different concentrations of isolated SARS-COV-2 RNA (3 samples in each concentration) have been carried out for RT-qPCR reactions untiled the viral cDNA could be detected. The measured Cq values for different concentrations of isolated RNA samples are shown in FIG. 10.

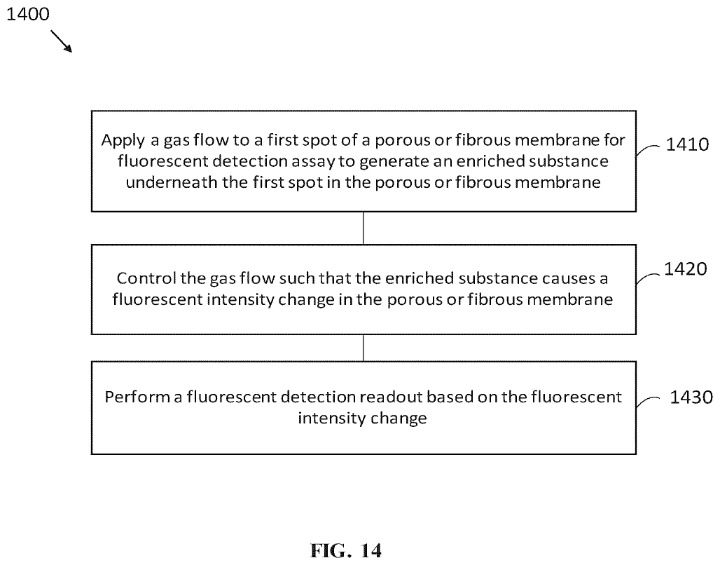

FIG. 14 shows an example method of colorimetric detection assay based on some embodiments of the disclosed technology.

In some implementations, a method 1400 includes, at 1410, a gas flow to a first spot of a porous or fibrous membrane for fluorescent detection assay to generate an enriched substance underneath the first spot in the porous or fibrous membrane, at 1420, controlling the gas flow such that the enriched substance causes a fluorescent intensity change in the porous or fibrous membrane, and at 1430, performing a fluorescent detection readout based on the fluorescent intensity change.

Therefore, various implementations of features of the disclosed technology can be made based on the above disclosure, including the examples listed below.
Example 1. A method comprising: applying a gas flow to a first spot of a porous or fibrous membrane for fluorescent detection assay to generate an enriched substance underneath the first spot in the porous or fibrous membrane; controlling the gas flow such that the enriched substance causes a fluorescent intensity change in the porous or fibrous membrane, and performing a fluorescent detection readout based on the fluorescent intensity change.
Example 2. The method of example 1, wherein the gas flow is controlled in a static mode to generate an evaporative gradient without moving the first spot and to generate fluid flows in the porous or fibrous membrane that are directed radially inward.
Example 3. The method of example 1, wherein the gas flow is controlled in a dynamic mode to allow a transportation of a solute to specified positions along a surface of the porous or fibrous membrane by moving the first spot of the porous or fibrous membrane.
Example 4. The method of example 1, wherein the porous or fibrous membrane includes a pre-wetted membrane to generate an evaporative gradient such that a solute in the porous or fibrous membrane becomes enriched underneath the first spot.
Example 5. The method of example 1, wherein the enriched substance includes a colorimetric substance.
Example 6. The method of example 1, wherein the fluorescent detection assay includes a colorimetric detection assay for nucleic acids and proteins via loop-mediated isothermal amplification (LAMP) and enzyme linked immunosorbent assay (ELISA).
Example 7. The method of example 1, wherein the gas flow includes a nitrogen gas.
Example 8. The method of example 1, wherein the gas flow is applied to the first spot of the porous or fibrous membrane using an airflow nozzle placed perpendicular to the porous or fibrous membrane.
Example 9. The method of example 1, further comprising adjusting at least one of a temperature or a speed of the air flow.
Example 10. A device comprising: a pad including a central portion that includes a liquid and a constituent in the liquid, wherein an evaporation rate of the liquid increases with a gas flow over the central portion, wherein the constituent has a lower evaporation rate than the liquid under the same gas flow rate, and a nozzle positioned to allow one or more gases to pass through and positioned at a distance above the central portion of the pad.
Example 11. The device of example 10, wherein the pad includes a porous or fibrous membrane
Example 12. The device of example 10, wherein the pad includes a paper.
Example 13. The device of example 10, wherein the one or more gases include a nitrogen gas.
Example 14. A device comprising a hollowed pad including a hole portion at a center of the pad and a replenishing pad portion surrounding the hole portion; and a nozzle structured to allow one or more gases to pass through and positioned at a distance above the hollowed pad
Example 15. The device of example 14, further comprising: a filter cutout to accommodate a sample to be enriched, the filter cutout positioned on the hollowed pad, wherein the filter cutout has a larger area than the hole portion of the hollowed pad such that a perimeter of the filter cutout overlaps at least part of the replenishing pad portion adjacent to the hole portion, wherein the nozzle is positioned perpendicular to the filter cutout and configured to apply the one or more gases to a first spot of the filter cutout.
Example 16. The device of example 15, wherein the filter cutout has a circular shape, and when positioned on the hollowed pad, is concentric with the hole portion of the hollowed pad.
Example 17. The device of example 15, wherein a contact between different materials is only made at the perimeter of the filter cutout.
Example 18. The device of example 15, wherein the hollowed pad includes a blotting paper saturated with water.
Example 19. The device of example 15, further comprising: a platform including a hollow section and configured to support the hollowed pad on the platform
Example 20. The device of example 15, wherein the gases include a nitrogen gas.
Example 21. A device comprising: a fibrous membrane: a liquid in the fibrous membrane, wherein the evaporation rate of the liquid increases with a gas flow, a constituent in the liquid that is moved towards a position of the gas flow; and a nozzle positioned to introduce a gas flow to a segment of the fibrous membrane.
Example 22. The device of example 21, further comprising: a sensor configured to detect a color change of an area under a flow of the gases.
Example 23. The device of example 21, wherein an evaporation rate of the liquid increases with a gas flow, wherein the constituent has a lower evaporation rate than the liquid under the same gas flow rate.
Example 24. The device of example 21, wherein the fibrous membrane includes a central area, and wherein the nozzle is positioned above the central area to direct an air flow to the central area.
Example 25. The device of example 24, further comprising: a replenishing area connected to the central area to allow liquid flow from the replenishing area to the central area.
Example 26. The device of example 25, wherein the liquid flow includes the constituent.
Example 27. A device comprising a fibrous membrane including a portion that includes a liquid and a constituent therein, wherein an evaporation rate of the liquid increases with a gas flow over the portion, wherein the constituent has a lower evaporation rate than the liquid under the same gas flow rate; a nozzle positioned to allow one or more gases to pass through and positioned at a distance above the central portion of the pad; and a sensor configured to detect a color change of an area under a flow of the one or more gases
Example 28. A device comprising: a fibrous membrane; a liquid in the fibrous membrane, wherein the evaporation rate of the liquid increases with a gas flow; a constituent in the liquid that is moved towards a position of the gas flow; and a nozzle positioned to introduce a gas flow to a segment of the fibrous membrane.
Example 29. A device comprising a fibrous membrane including a central area; a replenishing area connected to the central area to allow liquid flow from the replenishing area to the central area; and a nozzle positioned above the central area to direct an air flow to the central area, wherein the liquid flow includes a constituent that is moved towards a position of the air flow.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A method comprising:
applying a gas flow to a first spot of a porous or fibrous membrane for fluorescent detection assay to generate an enriched substance underneath the first spot in the porous or fibrous membrane;
controlling the gas flow such that the enriched substance causes a fluorescent intensity change in the porous or fibrous membrane; and
performing a fluorescent detection readout based on the fluorescent intensity change.

2. The method of claim 1, wherein the gas flow is controlled in a static mode to generate an evaporative gradient without moving the first spot and to generate fluid flows in the porous or fibrous membrane that are directed radially inward.

3. The method of claim 1, wherein the gas flow is controlled in a dynamic mode to allow a transportation of a solute to specified positions along a surface of the porous or fibrous membrane by moving the first spot of the porous or fibrous membrane.

4. The method of claim 1, wherein the porous or fibrous membrane includes a pre-wetted membrane to generate an evaporative gradient such that a solute in the porous or fibrous membrane becomes enriched underneath the first spot.

5. The method of claim 1, wherein the enriched substance includes a colorimetric substance.

6. The method of claim 1, wherein the fluorescent detection assay includes a colorimetric detection assay for nucleic acids and proteins via loop-mediated isothermal amplification (LAMP) and enzyme linked immunosorbent assay (ELISA).

7. The method of claim 1, wherein the gas flow includes a nitrogen gas.

8. The method of claim 1, wherein the gas flow is applied to the first spot of the porous or fibrous membrane using an airflow nozzle placed perpendicular to the porous or fibrous membrane.

9. The method of claim 1, further comprising:
adjusting at least one of a temperature or a speed of the gas flow.

10. A device comprising:
a pad including a central portion that includes a liquid and a constituent in the liquid, wherein an evaporation rate of the liquid increases with a gas flow over the central portion, wherein the constituent has a lower evaporation rate than the liquid under a same gas flow rate; and
a nozzle positioned to allow one or more gases to pass through and positioned at a distance above the central portion of the pad.

11. The device of claim 10, wherein the pad includes a porous or fibrous membrane.

12. The device of claim 10, wherein the pad includes a paper.

13. The device of claim 10, wherein the one or more gases include a nitrogen gas.

14. A device comprising:
a hollowed pad including a hole portion at a center of the pad and a replenishing pad portion surrounding the hole portion; and
a nozzle structured to allow one or more gases to pass through and positioned at a distance above the hollowed pad.

15. The device of claim 14, further comprising:
a filter cutout to accommodate a sample to be enriched, the filter cutout positioned on the hollowed pad, wherein the filter cutout has a larger area than the hole portion of the hollowed pad such that a perimeter of the filter cutout overlaps at least part of the replenishing pad portion adjacent to the hole portion, wherein the nozzle is positioned perpendicular to the filter cutout and configured to apply the one or more gases to a first spot of the filter cutout.

16. The device of claim 15, wherein the filter cutout has a circular shape, and when positioned on the hollowed pad, is concentric with the hole portion of the hollowed pad.

17. The device of claim 15, wherein a contact between different materials is only made at the perimeter of the filter cutout.

18. The device of claim 15, wherein the hollowed pad includes a blotting paper saturated with water.

19. The device of claim 15, further comprising:
a platform including a hollow section and configured to support the hollowed pad on the platform.

20. The device of claim 15, wherein the gases include a nitrogen gas.

21-26. (canceled)
